(19) United States
(12) Patent Application Publication
(10) Pub. No.: US 2020/0035332 A1
(43) Pub. Date: Jan. 30, 2020

(54) METHOD AND APPARATUS FOR MASKING CLINICALLY IRRELEVANT ANCESTRY INFORMATION IN GENETIC DATA

(71) Applicant: KONINKLIJKE PHILIPS N.V., EINDHOVEN (NL)

(72) Inventor: Vartika Agrawal, Cambridge, MA (US)

(21) Appl. No.: 16/500,459

(22) PCT Filed: Apr. 4, 2018
(86) PCT No.: PCT/EP2018/058650

§ 371 Date: Oct. 3, 2019

Related U.S. Application Data
(60) Provisional application No. 62/482,364, filed on Apr. 6, 2017.

(51) Int. Cl. G16B 50/40; G16B 20/20; G06F 21/62

(52) U.S. Cl. CPC G16B 50/40; G06F 21/6254; G16B 20/20

(57) ABSTRACT

Methods and corresponding systems for anonymizing genetic data obtained from a patient are described. The ancestry data can be masked by identifying ancestry information marker (AIM) regions in the genetic data. Each AIM region can include including one or more single-nucleotide polymorphism (SNP) alleles associated with a population of patients belonging to a certain ancestry. Once the AIM regions are identified, one or more regions that include clinically relevant data can be identified. The clinically relevant data can be data having one or more gene variants associated with a specific disease or disorder. The genetic data can be anonymized the by masking or removing AIM regions that do not include clinically relevant data.

### FIELD

The present disclosure generally relates to method and systems for anonymizing genetic data obtained from a patient. More specifically, the present disclosure relates to identifying ancestry information marker (AIM) regions, in the genetic data of a patient, which can associate the patient with a population of patients belonging to a certain ancestry, and anonymizing the genetic data by making or removing AIM regions that do not include clinically relevant data.

### BACKGROUND

Maintaining patient privacy is among the challenges faced by researchers that use clinical patient data in genomic research. Since genetic data can include information about the patient's ancestry, such data can reveal ancestry specific information including propensity for developing genetic diseases.

Although techniques for anonymizing genetic data and performing secure processing and computing exist, even anonymized patient data can include intrinsic information regarding that patient's ancestry. The ancestry information included patient data can, for example, reveal a potential propensity for developing a certain disease or disorder. Availability of such information can lead to discriminatory practices against individuals belonging to the ancestry linked with the disease or disorder. For example, insurance companies can use this information to discriminate against the individuals belonging to that ancestry, deny coverage to them, or require them to pay higher premiums for coverage.

### SUMMARY

In one aspect, a method for anonymizing genetic data obtained from a patient is featured. The featured method includes:
identifying one or more ancestry information marker (AIM) regions in the genetic data, each AIM region including one or more single-nucleotide polymorphism (SNP) alleles associated with a population of patients belonging to a certain ancestry;
identifying one or more regions, from among the one or more AIM regions, that include clinically relevant data, the clinically relevant data being data including one or more gene variants associated with a specific disease or disorder;

anonymizing the genetic data by masking or removing AIM regions that do not include clinically relevant data; and
reporting the anonymized genetic data to a user.

In another aspect, a data processing system is described. The system comprises at least one memory operable to store a data repository, and a processor communicatively coupled to the at least one memory. The processor is operable to:
identify one or more ancestry information marker (AIM) regions in genetic data obtained from a patient, each AIM region including one or more single-nucleotide polymorphism (SNP) alleles associated with a population of patients belonging to a certain ancestry;
identify one or more regions, from among the one or more AIM regions, that include clinically relevant data, the clinically relevant data being data including one or more gene variants associated with a specific disease or disorder;
anonymize the genetic data by masking or removing AIM regions that do not include clinically relevant data; and
report the anonymized genetic data to a user.

In yet another aspect, a computer program product is described. The computer program product is tangibly embodied in a non-transitory computer readable storage medium that comprises instructions being operable to cause a data processing system to:
identify one or more ancestry information marker (AIM) regions in genetic data obtained from a patient, each AIM region including one or more single-nucleotide polymorphism (SNP) alleles associated with a population of patients belonging to a certain ancestry;
identify one or more regions, from among the one or more AIM regions, that include clinically relevant data, the clinically relevant data being data including one or more gene variants associated with a specific disease or disorder;
anonymize the genetic data by masking or removing AIM regions that do not include clinically relevant data; and
report the anonymized genetic data to a user.

In other examples, any of the above aspects, or any system, method, apparatus, and computer program product method described herein, can include one or more of the following features.

The SNP alleles can differentiate the patients belonging to the certain ancestry from patients belonging to other ancestries. The patients belonging to the certain ancestry can include patients having at least one of same or similar race, ethnicity, religious background, skin color, or country of origin.

One or more AIM regions that include the clinically relevant data can be identified in response to the user's request for genetic data relating to the specific disease or disorder. In an event one or more AIM regions that include clinically relevant data are identified, confirmation from the user indicating that the user is authorized to access the genetic data can be requested such that the data can be reported to the user upon receiving the confirmation.

The genetic data can include gene annotations identifying locations of genes or gene variants and their possible associations with various diseases or disorders. The one or more AIM regions that include clinically relevant data can be identified using the gene annotations. Each gene or gene variant associated with the specific disease or disorder can be divided into one or more classes of genes or gene variants based on a probability that the gene or gene variant triggers the specific disease or disorder. The user can be required provide various levels of authorization for accessing data having the AIM regions that include the clinically relevant data based on the class of gene or gene variant to which the clinically relevant data belongs. Data Regions other than clinically relevant regions can be removed from the anonymized genetic data.

The user can be a clinician who is making a clinical determination relating to the specific disease or disorder.

Other aspects and advantages of the invention can become apparent from the following drawings and description, all of which illustrate the principles of the invention, by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 is a high-level block diagram of a system for masking clinically irrelevant ancestry information in genetic data according to embodiments described herein.

FIG. 2 is a high-level block diagram of labeled genomic data that can be used with the embodiments described herein.

FIG. 3 is an example of procedures for masking ancestry informative markers according to an embodiment described herein.

FIG. 4 is an example of procedures for masking ancestry informative markers according to an embodiment described herein.

### DETAILED DESCRIPTION

Maintaining privacy of patient is an important concern to researchers using clinical genetic data. Since a patient's genetic data can include information about that person's ancestry, the patient's genetic data can reveal possible propensities of people of the same ancestry for contracting or developing certain genetic conditions or diseases. This information can be harmful to the patient and those having the same ancestry as the patient because it can lead to discriminatory practices against the patient or those having the same ancestry. For example, insurance companies may use such information to discriminate against people of that ancestry, deny coverage to such individuals, or require them to pay higher premium that others.

Maintaining the privacy and security of genomic data is also important in other facets of healthcare (e.g., diagnosis, prognosis and therapy guiding) because the genomic data can potentially be used for undesirable purposes. For example, as noted, since genetic data can reveal a potential propensity for developing certain genetic disorders, insurance companies may be able to use this data to discriminate against the individuals belonging to ancestries linked with genetic disease. Genetic data can also reveal information about families and ethnic heritages that can be potentially harmful to the families and ethnic heritages. Further, genetic data can reveal vital information about an individual's family members and create consent issues. For example, consent issues may arise in situations in which a person has agreed to the use of her genetic information but her relatives/family members have not.

Accordingly, genomic privacy is an important factor when genomic data is used in healthcare delivery. Embodiments described herein reduce the risk of retracing a patient's identity based on her genomic data by leveraging the fact that certain parts of a person's genome, commonly referred to as Ancestry Information markers (AIMs), are often not clinically significant but can reveal the person's ancestry. Although ancestry information, alone, cannot easily reveal the person's identity, the combination of ancestry information and some other information, such as the person's zip code, can be used to narrow down and possibly identify the person.

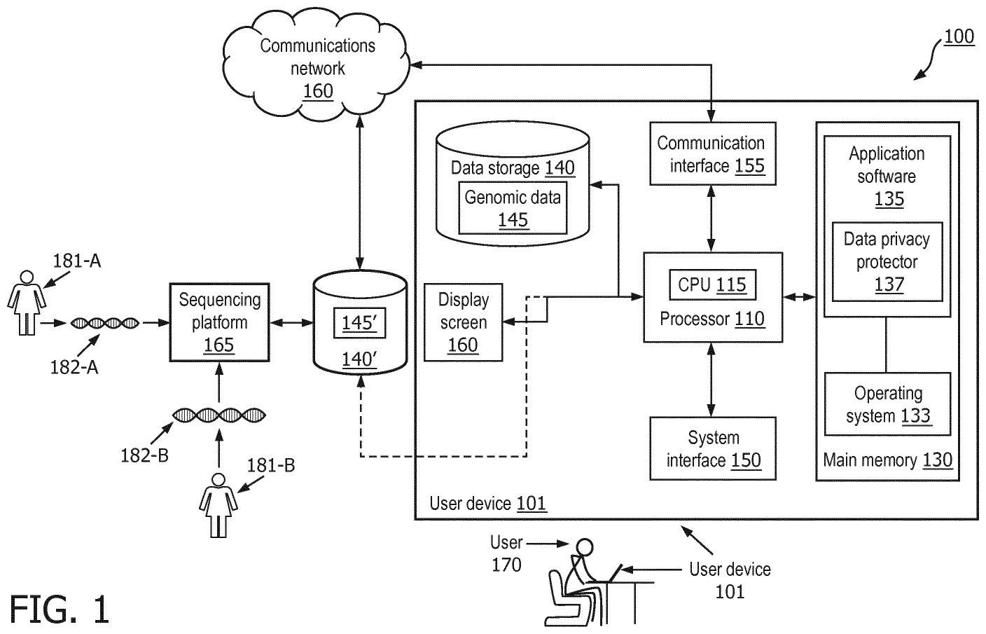

FIG. 1 is a high-level block diagram of an ancestry data masking system 100 according to an embodiment described herein. Although in the example shown in FIG. 1, the ancestry data masking system 100 is shown as having been implemented in an interactive user device 101 (e.g., computer). However, the system 100 can be a computer implemented system and/or be implemented in digital electronic circuitry or computer hardware.

The user device 101 can be any device that includes a processor capable of carrying and/or implementing the procedures described herein. For example, the user device 101 can be a wireless phone, a smart phone, a personal digital assistant, a desktop computer, a laptop computer, a tablet computer, a handheld computer, a workstations, etc. Further, as noted above, one skilled in the art should appreciate that the system 100 can be implemented using any techniques known in the art, for example on an electronic chip.

In the example shown in FIG. 1, the user device 101 that implements the system 100 includes a main memory 130 having an operating system 133. The main memory 130 and the operating system 133 can be configured to implement various operating system functions. For example, the operating system 133 can be responsible for controlling access to various devices, implementing various functions of the user device 101, and/or memory management. The main memory 130 can be any form of non-volatile memory included in machine-readable storage devices suitable for embodying data and computer program instructions. For example, the main memory 130 can be magnetic disk (e.g., internal or removable disks), magneto-optical disks, one or more of a semiconductor memory device (e.g., EPROM or EEPROM), flash memory, CD-ROM, and/or DVD-ROM disks.

The main memory 133 can also hold application software 135. For example, the main memory 130 and application software 135 can include various computer executable instructions, application software, and data structures such as computer executable instructions and data structures that implement various aspects of the embodiments described herein. For example, the application software 135 can include various computer executable instructions, application software, and data structures such as computer executable instructions and data structures that implement the data privacy protector 137 described herein.

The main memory 130 can also be connected to a cache unit (not shown) configured to store copies of the data from the most frequently used main memory 130. The program codes that can be used with the embodiments disclosed herein can be implemented and written in any form of programming language, including compiled or interpreted languages, and can be deployed in any form, including as a stand-alone program or as a component, module, subroutine, or other unit suitable for use in a computing environment. A computer program can be configured to be executed on a computer, or on multiple computers, at one site or distributed across multiple sites and interconnected by a communications network 160.

The networks 160 can have various topologies (e.g., bus, star, or ring network topologies) and/or be a private network (e.g., local area network (LAN)), a metropolitan area network (MAN), a wide area network (WAN), or a public network (e.g., the Internet). The network 160 can be a hybrid communications network 160 that includes all or parts of other networks.

Further, as noted above, the techniques described herein, without limitation, can be implemented in digital electronic circuitry or in computer hardware that executes software, firmware, or combinations thereof. The implementation can be as a computer program product, for example a computer program tangibly embodied in a non-transitory machine-readable storage device, for execution by, or to control the operation of, data processing apparatus, for example a computer, a programmable processor, or multiple computers.

One or more programmable processors can execute a computer program to operate on input data, perform function and methods described herein, and/or generate output data. An apparatus can be implemented as, and method steps can also be performed by, special purpose logic circuitry, such as a field programmable gate array (FPGA) or an application specific integrated circuit (ASIC). Components can refer to portions of the computer program and/or the processor or special circuitry that implements that functionality.

The user device 101 can also include a processor 110 that implements the various functions and methods described herein. The processor 110 can be connected to the main memory 130. The processor 110 and the main memory 130 can be included in or supplemented by special purpose logic circuitry.

The processor 110 can include a central processing unit (CPU) 115 that includes processing circuitry configured to manipulate data structures from the main memory 130 and execute various instructions. For example, the processor 110 can be a general and/or special purpose microprocessor and any one or more processors of any kind of digital computer. Generally, the processor 110 can be configured to receive instructions and data from the main memory 130 (e.g., a read-only memory or a random access memory or both) and execute the instructions. The instructions and other data can be stored in the main memory 130.

The processor 110 can also be connected to various interfaces via a system interface 150, which can be an input/output (1/0) device interface (e.g., USB connector, audio interface, FireWire, interface for connecting peripheral devices, etc.). The processor 110 can also be connected a communications interface 155. The communications interface 155 can provide the user device 101 with a connection to a communications network 160. Transmission and reception of data, information, and instructions can occur over the communications network 160.

The processor 110 can also be connected to a display 160 for receiving and/or displaying information (e.g., monitor, display screen, etc.). Although shown as an interactive system having a display, one of ordinary skill in the art should appreciate that the system 100 disclosed herein are not limited to embodiments implemented using a computer or implementation requiring direct interactions with a user. The system 100 can be implemented in chip or in any other electronic hardware known in the art and operate without requiring any interaction or feedback from a user 170.

The processor 110 can also be coupled to one or more data storage elements 140, 140′ and be arranged to transfer data to and/or receive data from the data storage elements 140, 140′. The data storage element 140, 140′ can hold genomic data 145, 145′, including any data or information obtained from human subjects.

The term “genomic data,” 145, 145′ as used herein, refers to, but is not limited to gene expression data. For example, the genomic data 145, 145′ can be sequencing data obtained from sequencing a genome. The term “data sequencing,” as used herein, is used in its ordinary context in the fields of genetics, genomics, and bioinformatics and can be performed by any method or technique known in the art.

The data 145, 145′ can be stored in the secured form (e.g., encrypted form) in the data storage 140, 140′. The data storage 140, 140′ can also be coupled with various security systems or structures for protecting the security and maintaining the privacy of the data 145, 145′. Any technique known in the art for maintaining the security and privacy of the data 145, 145′ can be used.

Although shown as having been included in the user device 101, one skilled in the art should appreciate that the data storage 140, 140′ and/or genomic data 145, 145′ need not be included in the user device 101. The data storage 140, 140′ and any storage component storing the genomic data 145 can be positioned in a remote (or independent) position from the user device 101 and/or the data privacy protector 137 and connect to the user device 101 and/or the data privacy protector 137 using any techniques known in the art. For example, as shown in FIG. 1, the data storage 140′ and any storage component storing the genomic data 145′ can connect to the user device 101 and/or the data privacy protector 137 through the communications network 160.

Generally, the genomic data 145, 145′ can include any quantitative data obtained from one or more human subjects 181-A, 181-B. The genomic data 145, 145′ can be obtained using any genomic data generation platforms and include physical and/or biological measurements relating to the patient's 181-A, 181-B genomic information. For example, the genomic data 145, 145′ can be data obtained using a genomic data generation platform such as RT-PCR, microarray sequencing, Bead Array microarray technology, proteomics, etc.

The genomic data can be obtained on one or more specific disease or disorders. For example, the genomic data can be obtained from on breast cancer or any other disease or disorder believed to be a genetic condition. The terms disease or disorder, as used herein, are intended to refer to their ordinary meaning. For example, the disease or disorder can be a genetic condition possibility resulting from one or more modifications, mutations, insertions, or deletions in the genome of a human individual.

The genomic data 145, 145′ can be pre-processed genomic data that includes one or more identifiers that designate ancestry information marker (AIM) regions in the genomic data. Each AIM region can include one or more single-nucleotide polymorphism (SNP) alleles associated with a population of patients belonging to a certain ancestry. Specifically, the data can include one or more haplotypes or haploid genotypes. A haplotype can include a group of genes or SNP alleles along a region of a chromosome that are inherited together from a single or common parent. A haplotype block or group can be a block or group or markers that share a common ancestor. As used herein, the terms haplotype block or group (haplogroup) refer to SNP or unique-event polymorphism (UEP) mutations that represent a common or specific ancestry, Glade, or population to which the patient 181-A, 181-B, from whom the SNP was obtained, can belong. Generally, haplotypes exists due to low recombination rates and can, therefore, serve as biomarkers to reveal individual ancestry. Genetic studies also seem to indicate that haplotype analysis can provide valuable lineage information about an individual.

For example, studies conducted on gene SLC24A5 have revealed that this gene appears to include three haplotypes exclusively belonging an Asian Population of patients. Such studies seem to indicate that SNPs in a specific gene can serve as important biomarkers in predicting the ancestry of individuals. In fact, given their apparent ability to differentiate some populations of humans from other populations of humans, these biomarkers are commonly termed as “Ancestry Informative Markers” (AIMs). Therefore, if “ancestry-informative” markers are selected such that they include large allele frequency differences between population groups, even anonymized personal genetic data can be used to reveal a person's ancestry.

In addition to potentially revealing a person's ancestry, certain SNPs can potentially reveal other information about the patient from whom the genomic data containing the SNPs were obtained. For example, the gene “SLC24A5” is commonly known to encode for a protein, known as “solute carrier family 24 member 5,” which appears to have a major influence on natural skin colour variation. Mutations in this protein, however, do not appear to be associated with any disease or physiological effects. Therefore, although the presence of this information can compromise some aspects of the privacy of the genome, masking the information associated with this gene is not expected to disrupt any clinical decision-making. Accordingly, such genes can be processed to remove or mask any information that can compromise privacy of their originating subjects, while leaving behind any data that can be used in clinical decision-making. Removal of ancestry information from the genomic data 145, 145′ can be performed as a pre-processing procedure that is conducted before the genomic data 145, 145′ is deposited and/or stored in the data storage 140, 140′. Alternatively and/or additionally, removal of AIM regions that do not include clinically insignificant regions can be performed on the data 145, 145′ stored in the data storage 140, 140′, prior to providing the a user 170 with the genomic data 145, 145′.

As noted, the genomic data 145, 145′ can include pre-processed data having labels that identify SNP alleles that have been associated exclusively with a certain population of individuals. Although the privacy data protector 137 can receive and use the pre-processed data, it can, alternatively or additionally, pre-process the data to identify such SNP alleles. For example, the privacy data protector 137 can identify any haplotypes included in the data that may be population specific. The privacy data protector 137 can, for example, identify these haplotypes by comparing the data 145, 145′ against publically available datasets of known population specific haplotypes and/or mining information from available research studies, such as Natural Language Processing techniques. The datasets including the known population specific haplotypes (not shown) can be stored on the user device 101, for example in the data storage 140. Alternatively and/or additionally such datasets can be stored in a remote location (for example remotely positioned data storage 140′) and accessed by the data privacy protector 137 using any techniques available in the art (e.g., through communications network 160).

Generally, the data privacy protector 137 can use any available database, dataset, or information that can assist in identifying population specific SNP alleles in the data 145, 145′. The privacy data protector 137 can further use any machine learning or pattern recognition techniques known in the art to identify the population specific SNP alleles.

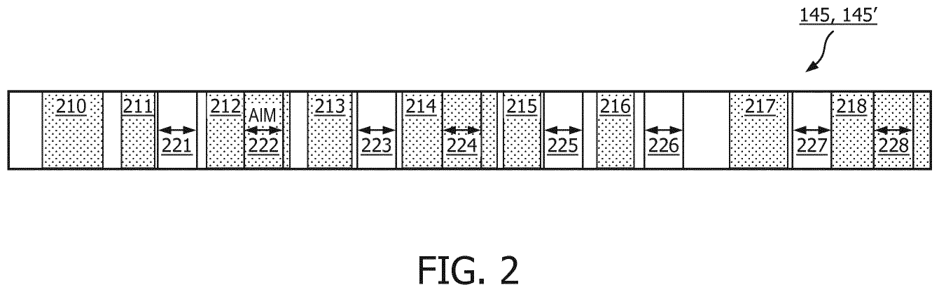

FIG. 2 is a high-level block diagram of the genomic data 145, 145′ that can be used with the embodiments described herein. The genomic data 145, 145′ can include one or more regions 210-218 that include clinically significant information. The data 145, 145′ can also include one or more AIM data regions 221-228 that include SNP alleles that have been associated exclusively with a certain population of individuals. As shown in FIG. 2, some data regions 221, 223, 225, 226, 227 can only include ancestry related information and be otherwise clinically irrelevant (do not include any clinically relevant information). The data 145, 145′ can also include regions 222, 224, 228 that include both ancestry related information and clinically relevant information. The data privacy protector 137, shown in FIG. 1, can process the data 145, 145′ to identify regions 221-228 that include ancestry related information. The data privacy protector 137 can further process the data 145, 145′ to identify ancestry regions 221-228 that also include clinically significant information (regions 222, 224, 228). Upon identification of these regions 222, 224, 228, the data privacy protector 137 can mask all ancestry related regions 221, 223, 225, 226, 227 that do not include any clinically significant information. In doing so, the data privacy protector 137 can use pre-processed data including labels identifying the ancestry related regions 221-228 and/or the clinically relevant regions 210-218.

The ancestry related regions can be labeled as ancestry information markers (AIMs). Ideally, all AIMS should be removed from the genomic data 145, 145′ to prevent the genomic data 145, 145′ from being traced back to the patient's ethnicity or ancestry. However, since a fraction of AIMS can include clinically relevant that provides valuable insights for the diagnosis, prognosis and therapy planning of the patient, the data privacy protector 137 only masks those AIMS that are shown to have no clinical relevance. The masking of AIMs can be done selectively or generally. Specifically, the data privacy protector 137 can selectively remove AIMs that are shown to have no clinical significance in the study for which the genomic data 145, 145′ is being requested. Alternatively or additionally, the data privacy protector 137 can generally remove all AIMs that are known not to have any clinical relevance. In doing so, the privacy data protector 137 can also identify the AIMs that are not clinically relevant. For example, as noted above, certain AIMs, such as gene “SLC24A5,” may commonly be known to encode for proteins that relate to people of common ancestries (e.g., skin color) but not have any known clinical significance. The privacy data protector 137 can remove such AIMs from the genomic data 145, 145′.

As noted, the genomic data 145, 145′ can also be pre-processed genomic data to include labels or markers indicating presence of clinically significant data. The labels or indicators can be designated to portions of the data that include genetic information pertaining to a specific disease or disorder. For example, the genomic data 145, 145′ can be data obtained on a specific disease or disorder, such as breast cancer. Further, as noted, the genomic data 145, 145′ obtained on a specific disease (e.g., breast cancer) can include labels that associate certain portions of the data with labels indicating that those data portions include genetic information pertaining to the specific disease. For example, the genomic data can include a label that indicates a certain portion (e.g., one or more data samples) of the breast cancer data includes the gene signature for the BRCA1 gene, which relates to certain types of breast cancer.

Once the AIMs that do not include clinically significant information have been identified, these regions are masked or removed from the genomic data 145, 145′ such that they cannot be used to trace back to the person's ancestry and/or ethnicity.

Generally, processing of genomic data 145, 145′, such as raw sequencing data obtained from sequencing platforms (e.g., next generation sequencing technologies), tends to generate actionable information and involve multiple stages (e.g., alignment, variant calling, variant annotation). Since each processing stage can generate various relevant data files, depending on the stage of data processing, masking can be performed in different manners.

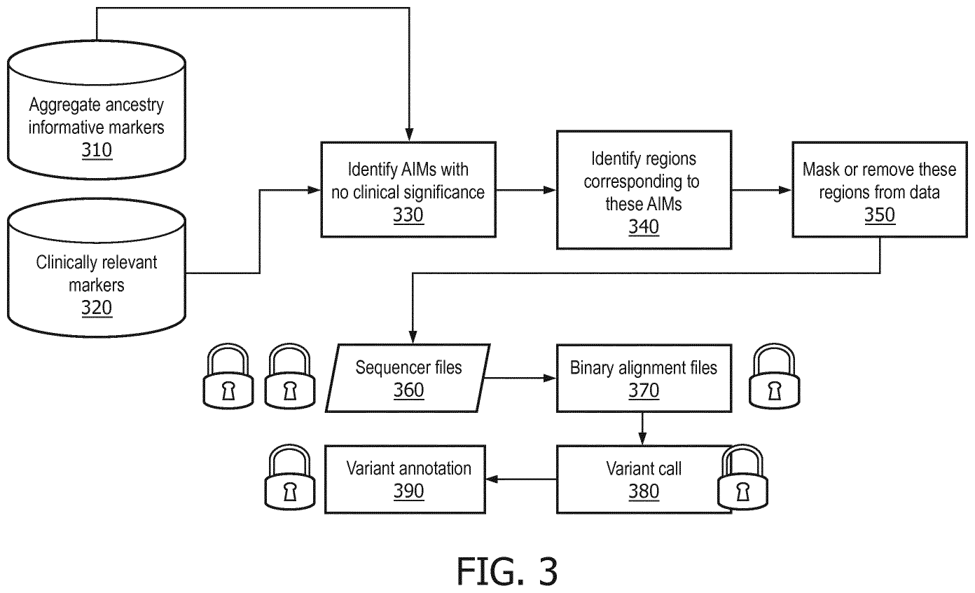

FIG. 3 is an example of procedures that can be used by the data privacy protector 137 to mask AIMs at an alignment level. One skilled in the art should appreciate that the term “alignment” refers to its ordinary usage in the fields of bioinformatics and gene sequencing. Generally, the term “alignment,” as used herein, refers to sequence alignment by arranging sequences of DNA, RNA, or protein to identify relationships (e.g., similarities) among sequences.

As shown in FIG. 3, information regarding aggregate ancestry informative markers (AIMs) 310 and clinically relevant markers 320 can be available and provided to the data privacy protector 137. For example, this information can be stored in one or more data storage structures 310, 320 and provided to the data privacy protector 137 for use in identifying AIM regions that are not clinically relevant. The data privacy protector 137 can compare the database of AIM markers 310 against the database of clinically relevant markers 320 to determine if there are any AIMs that do not have any known clinical significance 330. Once these clinically insignificant (regions with no clinical relevance) are identified, the data privacy protector 137 can determine the regions in the genomic data 145, 145′ that correspond to the AIMs identified as having no clinical relevance (or no clinical significance) 340. The regions of data corresponding to these clinically non-significant AIMs can then be masked or removed from the data 350.

Specifically, the data privacy protector 137 can use a database of aggregate ancestry informative markers 310 to identify markers that relate to a specific population and/or can be used to identify a specific population of individuals or people belonging to the same ancestry. The data privacy protector 137 can also obtain information regarding clinically relevant markers from a database 320 (that can be separate database from the database of the AIMs) that stores clinically relevant markers. For example, the privacy protector can store clinical markers for diseases or disorders, such as the clinical marker for breast cancer (e.g., BRCA, etc.).

As noted, once the AIMs and the clinically relevant markers are identified, the data privacy protector 137 can determine whether there are regions of data that correspond to an AIM but do not include any clinically significant information 340. Once such regions are specified, those regions can be masked or removed from the data 350.

The masking or removal of these regions of no clinical significance from the genomic data 145, 145′ can be done using any method or technique known in the art. The term “data masking,” as used herein, refers to its known and common use in the art and general field of maintaining privacy of data. Data masking can be done using any method or technique known in the art for maintaining the privacy of data. Data masks can be used to block access, transmission, or reading of the data. For example, the portion of data identified as corresponding to clinically non-significant SNPs can be encrypted such that it cannot be accessed. Additionally or alternatively, the portion of data including the clinically non-significant AIMs can be deleted, filtered, or removed from the data 145, 145′.

The information obtained from the AIM database 310 and the clinically relevant makers database 320 can then be applied to sequencer data files. The term “sequencer,” as used herein, is intended to refer to any platform or instrument that can be used in genetic sequencing. Generally, any sequencer known in the art that can analyze a genetic sample (e.g., DNA), determine the order of nucleobases in the sample, and report the order as a string (e.g., text string) can be used with the embodiments described herein. The term “read” is used hereinafter to refer to the output of a sequencer.

Generally, a sequencer files 360 can be any data file that includes genomic sequencing data. As shown in FIG. 3, sequencer files are often maintained under high security (shown by presence of two lock signs). For example, the genomic data can be encrypted to ensure its security. Generally, any method known in the art for maintaining the security and privacy of the genomic data can be used.

The data included in sequencer files 360 are often complex and require intensive computational power before they can be meaningful to users of genomic data. Binary alignment files 370 can be used to facilitate understanding these data files. The term “alignment,” as used herein, refers to its generally known meaning in the field of genomics and bioinformatics. Generally sequence alignment refers to aligning sequences of genomic data (obtained from genetic materials such as DNA, RNA, or protein) against a reference sequence to identify regions of similarity or variations between the sequences. More specifically, the term “alignment,” as used herein, refers to aligning a sequence of genomic data against a standard reference sequence that is expected to have similar properties as the sequence of genomic data and comparing the sequences to determine possible variations from the standard reference sequence. These variations are referred to hereinafter as “gene variants.” The term “gene variants,” as used herein, refers to the common meaning of this term in the art. Specifically, the term “gene variant” refers to a specific variation in a single nucleotide that occurs at a specific position in the genome. These variants can be germline variants, somatic mutations or de-novo. In many cases, a single gene variant may be sufficient to cause a genetic disease or disorder. Since as noted, the mutations can be inherited or new, both inherited abnormal mutations and new mutations can lead to a disease or disorder (e.g., haemophilia is caused by an inherited mutation while many cancers may be caused by new mutations).

As noted, variants existing in a sequence of genomic data can be identified by comparing the genomic data against a standard reference sequence and identifying differences (variants) that may exist between the sequence and the reference sequence. Since genomic data are often computationally complex, to help genomic data user gain a meaningful understanding of genomic data and the variations included in the data, identified variants are often annotated. Annotation of gene variants can facilitate understanding of genomic data and any variants that may be included in the data.

Variant annotation can be done using any technique or method known in the art. For example, information regarding AIM 310 and clinically relevant variants 320) can be stored or supplied to a user device 101, such as the user device 101 shown in FIG. 1. The data privacy protector 137 can use this information to generate annotation data for at least some (or possibly all) of variants included in files obtained from a sequencer 360.

Variant annotation 360 can be done in response to a variant call 380. Specifically, once the data privacy protector 137 receives a variant call 380, indicating the existence of a variant (nucleotide difference) at a given position, the data privacy protector 137 can respond by annotating the gene variants 390, analyzing the gene variant and identifying AIMs with no clinical significance from among the gene variant 330, identifying regions corresponding to AIMS that have no known clinical significance 340, and masking or removing these regions from the data 350.

The genomic data 145 can be de-identified data and the AIMS can be masked once an alignment file is generated using a modified reference. However, sequencer files 360 that are generated or processed before the AIM masking has begun can continue to have the AIM information and, thus, all steps to ensure the security of these files should be taken (noted by two locks next to the sequencer files box 360 in FIG. 3). The Binary Alignment file 370 and the variant calling file 380 may still have little information that could reveal the ancestry, and, thus, it is imperative to keep the file in a secure environment (depicted by 1 lock indicating a less stringent security level).

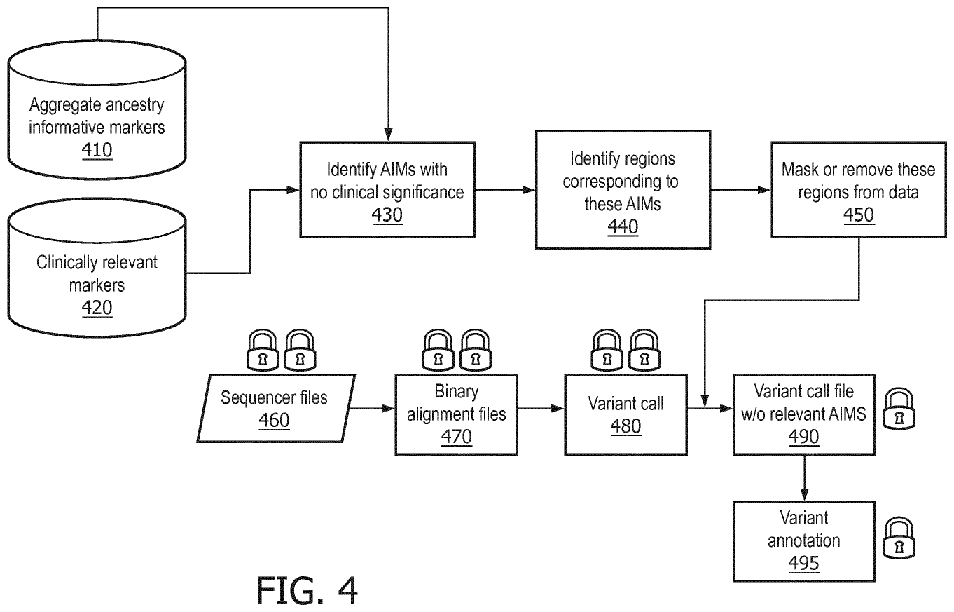

FIG. 4 is an example of procedures that can be used by the data privacy protector 137 to mask AIMs at a variant level. Similar to the embodiment described with respect to FIG. 3, the information regarding aggregate ancestry informative markers (AIMs) 410 and clinically relevant markers 420 can be available and provided to the data privacy protector 137. For example, this information can be stored in one or more data storage structures 410, 420 and provided to the data privacy protector 137 for use in identifying AIM regions that are not clinically relevant. The data privacy protector 137 can compare the database of AIM markers 410 against the database of clinically relevant markers 420 to determine if there are any AIMs that do not have any known clinical significance 430. Once these clinically insignificant markers (regions with no clinical relevance) are identified, the data privacy protector 137 can determine the regions in the genomic data 145, 145′ that correspond to the AIMs identified as having no clinical relevance (or no clinical significance) 440. The regions of data corresponding to these clinically non-significant AIMS can then be masked or removed from the data 450.

As noted, once the AIMs and the clinically relevant markers are identified, the data privacy protector 137 can determine whether there are regions of data that correspond to an AIM but do not include any clinically significant information 440. Once such regions are specified, those regions can be masked or removed from the data 450.

This removal of ancestry files can be performed at the variant level. Specifically, as shown in FIG. 4, sequencer files 460 can be used to generate binary alignment files 470. The term “alignment,” as noted refers to aligning a sequence of genomic data against a standard reference sequence that is expected to have similar properties as the sequence of genomic data and comparing the sequences to determine possible variations from the standard reference sequence. Once these variations are identified (variant call 480), the data privacy protector 137 can maintain any AIMs that are known to be clinically irrelevant in a separate file 490.

The embodiment shown in FIG. 4 can be used when dealing with de-identified genomic data. The AIMs can be masked once the variant file (e.g., VCF file) 490 is generated and it can be considered a “AIM devoid VCF file.”

Files that are generated or processed before the clinically irrelevant AIMs have been removed (e.g., BAM file and VCF file continue to have that information and, thus, all steps to ensure the security of these files should be taken (depicted by 2 locks). Erasing these files can also be considered as an option if data archiving is not necessary

The “AIM devoid VCF file” 490 may still have little information that could reveal the ancestry, and thus it is imperative to keep the file in a secure environment (depicted by 1 lock).

While the invention has been particularly shown and described with reference to specific illustrative embodiments, it should be understood that various changes in form and detail may be made without departing from the spirit and scope of the invention.

1. A method for anonymizing genetic data obtained from a patient, the method comprising:
identifying one or more ancestry information marker (AIM) regions in the genetic data, each AIM region including one or more single-nucleotide polymorphism (SNP) alleles associated with a population of patients belonging to a certain ancestry;
identifying one or more regions, from among the one or more AIM regions, that include clinically relevant data, the clinically relevant data being data including one or more gene variants associated with a specific disease or disorder;
anonymizing the genetic data by masking or removing AIM regions that do not include clinically relevant data; and
reporting the anonymized genetic data to a user.

2. The method of claim 1 wherein the SNP alleles differentiate the patients belonging to the certain ancestry from patients belonging to other ancestries.

3. The method of claim 1 wherein the patients belonging to the certain ancestry include patients having at least one of same or similar race, ethnicity, religious background, skin color, or country of origin.

4. The method of claim 1 further comprising identifying the one or more AIM regions that include the clinically relevant data in response to the user's request for genetic data relating to the specific disease or disorder.

5. The method of claim 1 further including:
in an event one or more AIM regions that include clinically relevant data are identified, requesting confirmation from the user indicating that the user is authorized to access the genetic data and reporting the data to the user upon receiving the confirmation.

6. The method of claim 1 wherein the genetic data include gene annotations identifying locations of genes or gene variants and their possible associations with various diseases or disorders.

7. The method of claim 6 further including identifying the one or more AIM regions that include clinically relevant data using the gene annotations.

8. The method of claim 7 further including dividing each gene or gene variant associated with the specific disease or disorder, into one or more classes of genes or gene variants, based on a probability that the gene or gene variant triggers the specific disease or disorder.

9. The method of claim 8 further including requiring the user to provide various levels of authorization for accessing data having the AIM regions that include the clinically relevant data based on the class of gene or gene variant to which the clinically relevant data belongs.

10. The method of claim 1 wherein the user is a clinician making a clinical determination relating to the specific disease or disorder.

11. The method of claim 1 further including removing, from the anonymized genetic data, data regions other than the clinically relevant regions.

12. A data processing system comprising:
at least one memory operable to store a data repository; and
a processor communicatively coupled to the at least one memory, the processor being operable to:
identify one or more ancestry information marker (AIM) regions in genetic data obtained from a patient, each AIM region including one or more single-nucleotide polymorphism (SNP) alleles associated with a population of patients belonging to a certain ancestry;
identify one or more regions, from among the one or more AIM regions, that include clinically relevant data, the clinically relevant data being data including one or more gene variants associated with a specific disease or disorder;
anonymize the genetic data by masking or removing AIM regions that do not include clinically relevant data; and
report the anonymized genetic data to a user.

13. A computer program product, tangibly embodied in a non-transitory computer readable storage medium, comprising instructions being operable to cause a data processing system to:
identify one or more ancestry information marker (AIM) regions in genetic data obtained from a patient, each AIM region including one or more single-nucleotide polymorphism (SNP) alleles associated with a population of patients belonging to a certain ancestry;
identify one or more regions, from among the one or more AIM regions, that include clinically relevant data, the clinically relevant data being data including one or more gene variants associated with a specific disease or disorder;
anonymize the genetic data by masking or removing AIM regions that do not include clinically relevant data; and
report the anonymized genetic data to a user.
